(19) United States
(12) Patent Application Publication
(10) Pub. No.: US 2011/0079582 A1
(43) Pub. Date: Apr. 7, 2011

(54) PLASMA GENERATING DEVICE AND METHOD

(76) Inventors: Akira Yonesu, Okinawa (JP); Nobuya Hayashi, Saga (JP)

(21) Appl. No.: 12/736,322

(22) PCT Filed: Mar. 31, 2009
(86) PCT No.: PCT/JP2009/056758

§ 371 Date: Dec. 20, 2010

(30) Foreign Application Priority Data
Mar. 31, 2008 (JP) 2008-094330

(51) Int. Cl. C23F 1/00; C23F 1/08; C23C 16/00; C23C 16/511; B01J 19/12; A61L 2/14

(52) U.S. Cl. 216/70; 156/345.38; 118/723 MA; 427/575; 422/186.01; 422/21

(57) ABSTRACT

An object of the invention is to provide a plasma generating device and method for generating plasma through electrodeless discharge within a long tubule and carrying out a plasma process on the inside of the long tubule. The plasma generating device has a container 1 for containing a long tubule 9, the internal pressure of which can be adjusted, a magnetic field applying means 8 for applying a magnetic field in at least part of the long tubule, and a microwave supplying means 2 for emitting microwaves into the container, and is characterized in that plasma is generated within the long tubule by emitting microwaves into the container in such a state that a magnetic field is applied in at least part of the long tubule.

### TECHNICAL FIELD

This invention relates to a plasma generating device and method, and in particular, to a plasma generating device and method which makes it possible to generate plasma in a long tubule to be sterilized or a long tubule of which the inner wall is to be coated with a film or etched using plasma.

### BACKGROUND ART

In recent years, various plasma processes, such as sterilizing processes, coating with a film and etching, have been used.

Some conventional methods for sterilizing a long tubule, such as a catheter or an endoscope use ultraviolet rays or high pressure steam, for example. However, such methods are inefficient in sterilization and, in some cases, the material may change in quality due to the ultraviolet rays or heat. Meanwhile, methods using an ethylene oxide gas or a liquid or gas of hydrogen peroxide are highly effective, but ethylene oxide and hydrogen peroxide are toxic, and hydrogen peroxide is particularly unsafe for those who handle it and dissolves clothing. In addition, in accordance with other methods, it is difficult to sufficiently sterilize the long tubule; liquids and gases other than ethylene oxide do not easily penetrate deep into the long tubule, due to their viscosity, nor do ultraviolet rays, due to their lack of transmissibility.

Thus, sterilizing methods using plasma has been proposed. In one such method, plasma is discharged between an external electrode and a central electrode formed in a discharge portion that is inserted into a long tubule, as in Patent Document 1. In accordance with this method, it is necessary to insert both the discharging portion and a power supplying wire for supplying power to the discharging portion into the long tubule.
Patent Document 1: Japanese Unexamined Patent Publication 2003-210556

However, it is difficult to provide a discharging portion having a small external and internal diameter, in order to avoid short-circuiting between the central electrode and the external electrode and secure a space for discharge between the two, and in addition, it is necessary to secure a certain thickness for the power supplying wires and the insulator between wires, in order to prevent the power supplying wires from causing insulation breakdown. Therefore, only thick tubules (having an inner diameter of 5 mm or more, preferably 1 cm or more, for example) can be sterilized, and in addition, the inner wall of the long tubule easily scratches when the discharging portion and the power supplying wires are inserted and removed. Furthermore, even after the sterilizing process, bacteria clinging to the surface of the discharging portion may stick to the inner wall, if the discharging portion or the power supplying wires make contact with it, thus causing secondary infection. In addition, in the case where the same sterilization processing device is used for different tubules, such secondary infection becomes a big problem.

The following Non-Patent Document 1 discloses a sterilization system for catheters that is carried out under atmospheric pressure. In this sterilization system, a wire electrode is inserted into a tubule and a plasma flow generated between the wire electrode and a grounding electrode outside the tubule.
Non-Patent Document 1: TOPICS “Development of sterilization system using atmospheric pressure non-equilibrium plasma flow,” Journal of the Japan Society of Mechanical Engineers, Vol. 110, No. 1063, p. 56, June 2007.

However, inserting a wire electrode into a tubule risks infecting or otherwise damaging the inside of the tubule, as with the above described Patent Document 1. In addition, the wire electrode may be sputtered by the plasma, and the metal that forms the electrode may adhere inside the tubule over the entire length thereof, and thus there is also a risk of the tubule being contaminated.

Furthermore, plasma generation is highly localized inside the tubule, between the wire electrode and the grounding electrode. Therefore, in the case where the tubule or electrode is moved in order to sterilize the entirety of the tubule, a moving mechanism is required, making the structure complex and increasing the risk of the tubule being damaged, as well as that of secondary infection. In addition, in the case where the grounding electrode is in cylindrical form and provided so as to surround the tubule, it is necessary to prepare cylindrical grounding electrodes of different diameters for tubules of various diameters. In addition, the tubule and grounding electrode are provided in close proximity to each other, and therefore there is a risk of the outside of the tubule being damaged or infected. Even if the diameter of the cylindrical grounding electrode is large, so that tubules of various diameters can be sterilized, the wire electrode and the grounding electrode are far apart, making the voltage applied across the electrodes high, and as a result, the wire electrode or grounding electrode is sputtered by the plasma, and the risk of the tubule being contaminated becomes high.

In order to solve the above described problems, Saga University, one of the present applicants, has proposed a plasma sterilizing device having a container for a long tubule to be sterilized, the pressure inside of which can be adjusted, and an electrode provided either inside or outside the long tubule, characterized in that an alternating current voltage is applied to the electrode in such a state that the pressure inside or outside of the long tubule is adjusted so that there is a predetermined difference in pressure between the inside and outside, and thus plasma is generated within the long tubule (see the following Patent Document 2).
Patent Document 2: Japanese Patent Application 2007-203559 (filed on Aug. 3, 2007)

The plasma sterilizing device disclosed in Patent Document 2 can generate plasma stably inside the long tubule, and therefore is excellent as a sterilizing means. However, it is necessary to place an electrode at one end of the long tubule, which takes time and effort, and in addition, it takes time to carry out a sterilizing process on a large number of tubules.

Plasma can be used for various other processes; for example, to etch or coat the surface of objects with a film. Concretely, a gas containing a titanium compound may be converted to plasma, which is then used to form a titanium film on the surface of the object. When a methane gas is introduced, a carbon film can be used in a plasma process. Furthermore, a plasma process may be carried out on a gas containing alcohol in order to form a hydrophilic organic film.

In the case where the object to be processed is a long tubule, however, it is difficult to generate plasma stably, and therefore it is impossible to etch or coat the inside of the long tubule with a film using plasma. In addition, in the case where an electrode is inserted into a long tubule, the electrode may damage the inside of the long tubule, or the electrode material may adhere to the inner wall, which is thus contaminated, as in the above described sterilizing process.

### DISCLOSURE OF THE INVENTION

### Problem to Be Solved by the Invention

An object of the present invention is to solve the above described problems and provide a plasma generating device and method for generating plasma through electrodeless discharge within a long tubule and carrying various processes using plasma within a long tubule.

### Means for Solving Problem

In order to achieve the above described object, the plasma generating device and method according to the present invention have the following characteristics.

(1) A plasma generating device having: a container for a long tubule, the pressure inside of which can be adjusted; a magnetic field applying means for applying a magnetic field in at least part of the long tubule; and a microwave supplying means for emitting microwaves into the container, characterized in that microwaves enter into the container in such a state that the pressure inside the long tubule is adjusted to a predetermined pressure that is higher than the pressure within the container, and a magnetic field is applied in at least part of the long tubule so that plasma is generated within the long tubule.
(2) The plasma generating device according to the above described (1), characterized in that the magnetic field applying means is formed of at lest a permanent magnet or an electromagnet.
(3) The plasma generating device according to the above described (2), characterized in that the magnetic field applying means is formed of a number of permanent magnets or electromagnets, and the magnets are arranged in such a manner that the same magnetic poles face each other.
(4) The plasma generating device according to any of the above described (1) to (3), characterized in that the pressure adjusting means for adjusting the pressure inside the container is formed so that it is possible to reduce the pressure within the container to 1 Pa or less.
(5) The plasma generating device according to any of the above described (1) to (4), characterized in that the long tubule is the object to be sterilized.
(6) The plasma generating device according to the above described (5), characterized in that a gas containing at least one of oxygen, argon and air is introduced into the container.
(7) The plasma generating device according to any of the above described (1) to (4), characterized in that the long tubule is the object the inner wall surface of which is to be coated with a film or etched using plasma.
(8) The plasma generating device according to any of the above described (1) to (5) and (7), characterized in that a gas is introduced directly into the long tubule.
(9) A plasma generating method, characterized in that: a long tubule is put in a container; and microwaves are emitted into the container in such a state that the inside of the long tubule is adjusted to a predetermined pressure that is higher than the pressure within the container and a magnetic field applied in at least part of the long tubule, and plasma is generated inside the long tubule.
(10) The plasma generating method according to the above described (9), characterized in that the pressure inside or outside the long tubule is adjusted so that there is a predetermined difference in pressure between the inside and outside the long tubule.
(11) The plasma generating method according to the above described (10), characterized in that the method for creating the difference in pressure is either a method for holding the long tubule in such a state that at least part of the long tubule is bent or a method for making the opening at an end of the long tubule narrow.
(12) The plasma generating method according to any of the above described (9) to (11), characterized in that at least part of a magnetic field applying means for applying a magnetic field is provided inside the container and the long tubule is provided so as to be wound around part of the magnetic field applying means.
(13) The plasma generating method according to any of the above described (9) to (11), characterized in that the magnetic field is a solenoid field or a mirror field and the long tubule is placed in the vicinity of the center of the magnetic field.
(14) The plasma generating method according to any of the above described (9) to (13), characterized in that the pressure within the long tubule is 0.01 Pa to 1 Pa when plasma is generated.
(15) The plasma generating method according to any of the above described (9) to (14), characterized in that the long tubule is the object to be sterilized.
(16) The plasma generating method according to the above described 15, characterized in that a gas containing at least one of oxygen, argon and air is introduced into the container.
(17) The plasma generating method according to the above described (15) or (16), characterized in that the input power for the microwaves is adjusted so that the temperature inside the long tubule remains at 60° C. or lower.
(18) The plasma generating method according to any of the above described (15) to (17), characterized in that the long tubule is contained in a resin bag for preventing bacteria and viruses from entering.
(19) The plasma generating method according to any of the above described (9) to (14), characterized in that the long tubule is an object the inner wall surface of which is to be coated with a film or etched using plasma.
(20) The plasma generating method according to any of the above described (9) to (15) and (19), characterized in that a gas is introduced directly into the long tubule.

### EFFECTS OF THE INVENTION

In accordance with the invention according to the above described (1), microwaves enter into the container containing a long tubule in such a state that the pressure inside the long tubule is adjusted to a predetermined pressure that is higher than the pressure within the container for containing the long tubule, and a magnetic field is applied in at least part of the long tubule, and thus plasma can be generated within the long tubule and it becomes possible to implement electrodeless discharge. Electrodeless discharge results from electron cyclotron resonance when electrons are accelerated by the microwaves in the magnetic field, in the case where the intensity of the magnetic field and the frequency of the microwaves have a certain relation under a certain air pressure.

Furthermore, according to the present invention, no electrode is inserted or attached to the long tubule, and therefore the long tubule cannot be damaged or contaminated. In addition, the electrode material is not sputtered and does not adhere to the long tubule, and therefore it becomes possible to carry out a plasma process in a highly safe and clean state.

In accordance with the invention according to the above described (2), the magnetic field applying means is formed of at least a permanent magnet or an electromagnet, and therefore, it is possible to select a magnetic field applying means which is appropriate for the type of plasma process.

In the case of a permanent magnet, for example, no driving circuit is required, the structure is simple, and no power is required to generate the magnetic field. Meanwhile, in the case of an electromagnet, it is possible to generate a magnetic field only at the time of the plasma process, and in addition, the intensity of the magnetic field is easy to adjust.

In accordance with the invention according to the above described (3), the magnetic field applying means is formed of a number of permanent magnets or electromagnets, and the magnets are arranged so that the same magnetic poles face each other, and therefore plasma rings can be generated around each magnet, and even in the case where the tubule is quite long, the tubule is easy to wind further around the magnetic field applying means.

In accordance with the invention according to the above described (4), the pressure adjusting means for adjusting the pressure within the container is formed so that the pressure within the container can be reduced to 1 Pa or less, and therefore it is possible to generate plasma through electronic cyclotron resonance.

In accordance with the invention according to the above described (5), a long tubule is the object to be sterilized, and therefore, bacteria that cling to the inner wall of the long tubule can be effectively decomposed and removed by generating plasma within the tubule. In addition, when plasma is generated, ultraviolet rays and radicals are also generated, in addition to electrons and ions. The sterilizing process for killing, or destroying, viruses and bacteria, as well as neutralizing, decomposing and removing proteins and lipids, including physical destruction of bacteria through ion impact in the plasma, destruction of DNA by ultraviolet rays and etching of the surface of bacteria by radical atoms and molecules, such as oxygen radicals and OH radicals, is effective using plasma and secondary substances, for example.

In accordance with the invention in accordance with the above described (6), a gas including at least one of oxygen, argon and air is introduced into the container, and therefore it is possible to improve the sterilizing effects, particularly in the case of oxygen, because oxygen radicals are generated. In the case of argon, it is possible to generate plasma outside the long tubule as well as inside, and thus it is possible to carry out a sterilizing process simultaneously on the inner wall and outer wall of the long tubule. In addition, air is the most inexpensive gas, and the present invention makes a sterilization process possible using it.

In accordance with the invention according to the above described (7), the long tubule is the object the inner wall surface of which is coated with a film or etched using plasma, and therefore it is possible to carry out a film coating or plasma etching process in a stable and clean state, by generating plasma within the long tubule.

In accordance with the invention according to the above described (8), a gas is introduced directly into the long tubule, and therefore it is very easy to adjust the pressure within the long tubule to a predetermined pressure.

In accordance with the invention according to the above described (9), plasma is generated within a long tubule by emitting microwaves into the container containing the long tubule in such a state that the pressure within the long tubule is adjusted to a predetermined pressure that is higher than the pressure within the container containing the long tubule and a magnetic field applied in at least part of the long tubule, and therefore it is possible to implement electrodeless discharge. In addition, as in the invention according to claim 1, the long tubule is not damaged, infected or contaminated because the discharge is electrodeless.

In accordance with the invention according to the above described (10), the pressure inside and outside the long tubule is adjusted so that there is a predetermined difference in pressure between the inside and outside of the long tubule, and therefore it is possible to generate plasma in either, and thus it is possible to selectively carry out the plasma process only inside or outside the long tubule.

In accordance with the invention according to the above describe (11), the method for creating a difference in pressure is either a method for holding the long tubule in such a sate that at least part of the long tubule is bent or a method for making the opening at one end of the long tubule narrow, and therefore it is easy to create a difference in pressure.

In accordance with the invention according to the above described (12), at least part of the magnetic field applying means for applying a magnetic field is placed in the container and the long tubule is provided so as to be wound around part of the magnetic field applying means, and therefore it is possible to apply a constant magnetic field throughout the entirety of the long tubule, and thus it becomes possible to generate plasma throughout the entirety of the long tubule.

In addition, it is possible to use part of the magnetic field applying means as a positioning means or holding means for positioning or holding the long tubule during the plasma process.

In accordance with the invention according to the above described (13), the magnetic field is a solenoid field or a mirror field and the long tubule is placed in the vicinity of the center of the magnetic field, and therefore it is possible to generate plasma in such a state that the long tubule is straight or folded, and thus it is possible to generate plasma in various types of long tubules with different lengths and flexibilities.

In accordance with the invention according to the above described (14), the pressure within the long tubule is 0.01 Pa to 1 Pa when plasma is generated, and therefore it is possible to generate plasma through electron cyclotron resonance.

In accordance with the invention according to the above described (15), the long tubule is the object to be sterilized, and therefore, it is possible to effectively decompose or remove bacteria that cling to the inner wall of the long tubule by generating plasma within the tubule. In addition, as with the invention according to claim 4, it is possible to use ultraviolet rays and radicals, in addition to plasma, for an efficient sterilizing process, and at the same time, the long tubule can be prevented from being damaged, infected or contaminated with an electrode material.

In accordance with the invention according to the above described (16), a gas including at least one of oxygen, argon and air is introduced into the long tubule, and therefore oxygen radicals can be generated, in the case of oxygen, and it becomes possible to generate plasma outside as well as inside the long tubule, in the case of argon, as in claim 5. Furthermore, the sterilizing process is inexpensive in the case of air.

In accordance with the invention according to the above described (17), the input power for the microwaves is adjusted so that the temperature inside the long tubule remains 60° C. or lower, and therefore it becomes possible to carry out a sterilizing process using plasma even in the case where the heat resistance is low; for example in the case where the object to be sterilized is made of a resin.

In accordance with the invention according to the above described (18), the long tubule is contained in a resin bag for preventing bacteria and viruses from entering, and therefore bacteria can be prevented from clinging to the long tubule after the sterilizing process, unlike in the case where there is no bag, and the long tubule remains sterilized as long as it remains in the bag.

In accordance with the invention according to the above described (19), The long tubule is the object the inner wall surface of which is coated with a film or etched using plasma, and therefore it is possible to carry out a film coating or plasma etching process in a stable and clean state, by generating plasma within the long tubule, as in the invention according to claim 6.

In accordance with the invention according to the above described (20), a gas is introduced directly into the long tubule, and therefore it is extremely easy to adjust the pressure within the long tubule to a predetermined pressure, and thus it is easy to control plasma generation.

### BRIEF DESCRIPTION OF THE DRAWINGS

FIG. 1 is a schematic diagram showing the plasma generating device according to the present invention;

FIG. 2 is a diagram showing how a long tubule is provided around a magnetic field applying means in cylindrical form;

FIG. 3 is a diagram showing how a long tubule is provided between two magnetic field applying means;

FIG. 4 is a diagram showing how a long tubule is provided inside a magnetic field applying means using solenoid;

FIG. 5 is a diagram showing how a number of magnetic field applying means are arranged;

FIG. 6 is a diagram showing how a long tubule is held within a resin bag;

FIG. 7 is a graph showing the waveforms of an alternating current voltage applied in the microwave generating portion;

FIG. 8 is a photograph showing the glow discharge within a long tubule;

FIGS. 9A and 9B are diagrams showing how a long tubule is arranged at a point where an optical magnetic field is generated around a magnet;

FIG. 10 is a diagram showing an example of a method for generating a magnetic field in accordance with the length of the long tubule when a number of magnets are in a straight line;

FIG. 11 is a diagram showing how a gas is introduced directly into a long tubule;

FIG. 12 is a diagram showing how a mirror field is created and a long tubule is placed at the center of the magnetic field; and

FIGS. 13A and 13B are diagrams illustrating a method for adjusting the openings at the ends of a long tubule.

### EXPLANATION OF SYMBOLS

1 Container
2 microwave supplying means
3 waveguide
4 gas supplying means
5, 7 gas pipe
6 pressure reducing means
8, 80, 82, 83 magnetic field applying means
9 long tubule
10 plate for holding magnetic field applying means
11 resin bag
12 opening
81, 84 line of magnetic force
85 solenoid
86 cylindrical member

### BEST MODE FOR CARRYING OUT THE INVENTION

The plasma generating device and method according to the present invention are described in detail below.

Though the below description focuses on a plasma sterilizing device and method, the plasma generating and method according to the present invention are naturally not limited to a sterilizing process.

### (Structure and Principle Behind Plasma Generating Device)

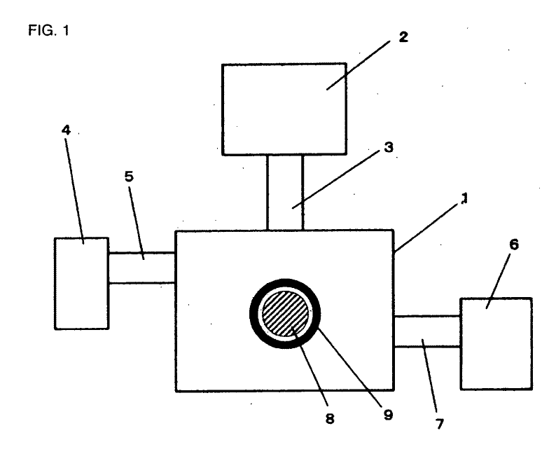

FIG. 1 is a schematic diagram showing the plasma generating device according to the present invention.

The plasma generating device according to the present invention has a container 1 for containing a long tubule 9 the pressure within which can be adjusted, a magnetic field applying means 8 for applying a magnetic field in at least part of the long tubule, and a microwave supplying means 2 for emitting microwaves into the container, and is characterized in that microwaves are emitted into the container in such a state that the pressure inside the long tubule is adjusted to a predetermined pressure that is higher than that within the container, and a magnetic field is applied in at least part of the long tubule and plasma is generated within the long tubule. Here, 3 is a waveguide for guiding the microwaves generated by the microwave supplying means 2 into the container 1. 4 is a gas supplying means for supplying a gas into the container, and 5 is a gas pipe for connecting the gas supplying means 4 to the container 1. 6 is a pressure reducing means for reducing the pressure within the container 1, and 7 is a gas pipe for connecting the pressure reducing means 6 to the container 1.

The present invention is characterized in that electrodeless discharge is possible. In particular, in the case where the intensity of the magnetic field and the frequency of microwaves have a certain relationship under a certain pressure, electrons accelerate in the magnetic field in a resonant manner, due to the microwaves, so that so-called electronic cyclotron resonance is generated, and plasma can be generated by the accelerated electrons. A magnetic field of 875 G is generated in the air under a pressure of 0.01 Pa to 1 Pa, for example, which is irradiated with microwaves of 2.45 GHz, so that plasma is generated through electronic cyclotron resonance.

Various types of gases can be introduced into the container 1 using the gas supplying means 4 in accordance with the object; for example, oxygen, argon or air can be used in the case of a sterilizing process.

In the case where oxygen is used, oxygen radicals are generated together with the plasma, and thus the sterilization effects improve.

In the case where argon is used, it becomes easy to generate plasma outside the long tubule, and therefore an appropriate sterilizing process can be carried out on the outer surface of the object to be sterilized.

Furthermore, air, which is an inexpensive gas, can be used for the sterilizing process using the plasma generating device and method according to the present invention.

Various types of gases, for example methane gas or alcohol, can be introduced into the long tubule when the inner wall surface of the long tubule is coated with a film. In addition, the etching gas can be selected in accordance with the material that forms the long tubule when etching it with plasma.

The pressure reducing means 6 is used to keep the gas in the container 1 at a constant pressure. It is necessary for the pressure reducing means to be able to reduce the pressure to 1 Pa or lower, in order to maintain the pressure within the container, particularly within the long tubule, at 0.01 Pa to 1 Pa. Concretely, a turbo pump or a cryo-pump can be used.

A magnetron is used for the microwave supplying means 2. As described below, the microwave supplying means is driven with pulses and the input power for the microwaves can be adjusted, and thus it becomes possible to adjust the temperature to which the object is heated by the plasma. The input power for generating continuous waves with a frequency of 2.45 GHz needs to be approximately 300 W in the case where the pressure within the container is 0.1 Pa and approximately 1 kW in the case of 0.5 Pa.

In addition, it is possible to emit only circularly polarized microwaves into the container via a mode converter. Though in this case the efficiency of plasma generation is high, the direction of emission and the like relative to the direction of the magnetic field is limited.

Next, the magnetic field applying means for applying a magnetic field to the long tubule is described.

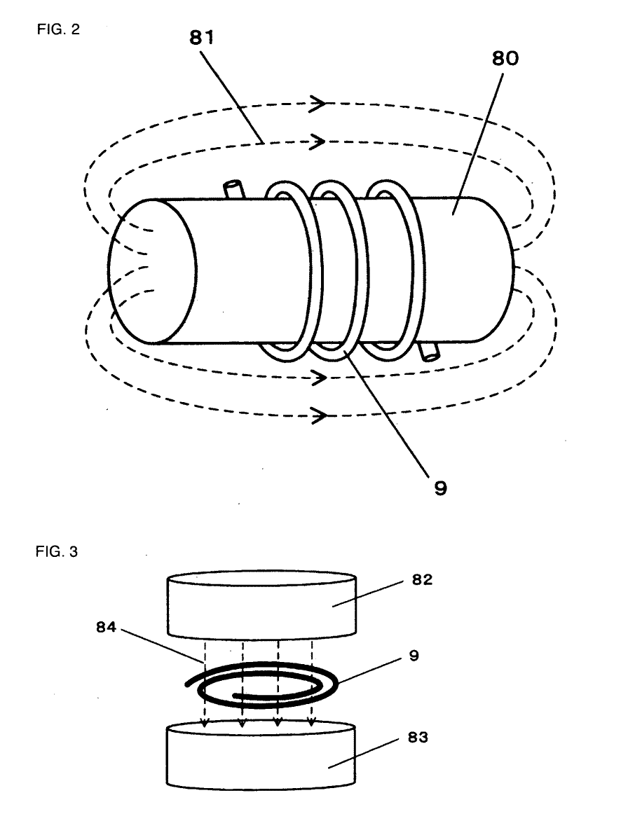

Various magnetic field applying means, such as permanent magnets and electromagnets, can be used in accordance with the application. In FIG. 2, a magnet 80 (permanent magnet or electromagnet) is placed within a container around which a long tubule is wound. Permanent magnets having a surface magnetic field of approximately 4000 G are appropriate, and such magnetic materials as neodymium or a samarium/cobalt alloy can be used.

It is not necessary for the long tubule to be wound around the container. In the case where a cylindrical magnet 80 is used, as in FIG. 2, however, lines of magnetic force 81, which are magnetic fields of the same intensity spread in concentric circles around center axis of the cylinder, and therefore, it is preferable for the long tubule to be wound around the magnet 80, in order to apply a magnetic field having the same intensity throughout the entire long tubule.

FIG. 3 shows an example of a magnetic field applying means where a long tubule 9 is provided between magnets 82 and 83. Magnetic fields of approximately the same intensity are generated in the space between the magnets 82 and 83 (lines of magnetic force 84). Therefore, it is not necessary for the long tubule 9 to be in circular or helical form.

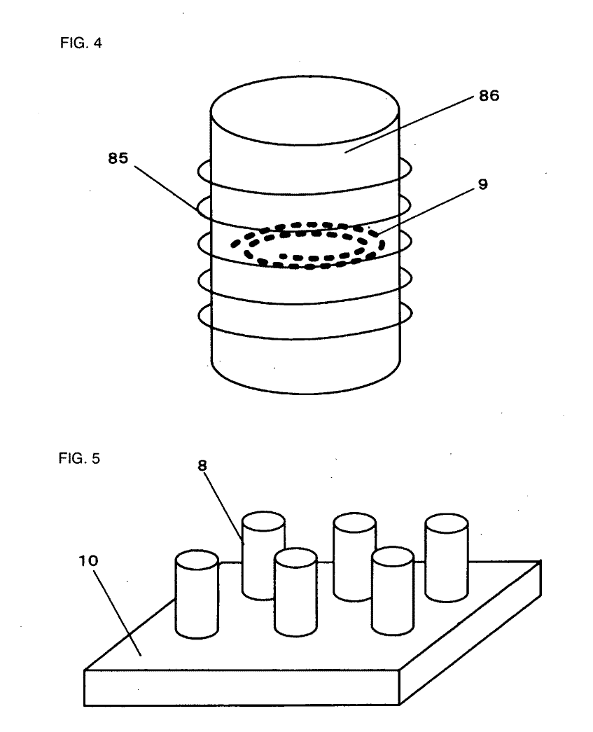

FIG. 4 shows a magnetic field applying means using an electromagnet having a solenoid. A solenoid 85 is formed around a cylindrical containing portion 86 through which a magnetic field can transmit, so that a uniform magnetic field is generated through the cylindrical containing portion 86 when a current flows through the solenoid. It is possible to apply a predetermined electrical field simply by providing a long tubule 9 within the cylindrical containing portion 86.

Though the container 1 contains only one magnetic field applying means 8 in FIG. 1, there may be more. As shown in FIG. 5, for example, a number of magnetic field applying means 8 are provided on a plate formed of a non-magnetic material, such as Teflon (registered trademark) so that a number of long tubules can be provided in each magnetic field applying means. Here, it is preferable for the magnetic field applying means 8 to be a certain distance apart, in order to prevent the magnetic fields from interfering with each other, and thus not prevent the magnetic field from being applied to the long tubules.

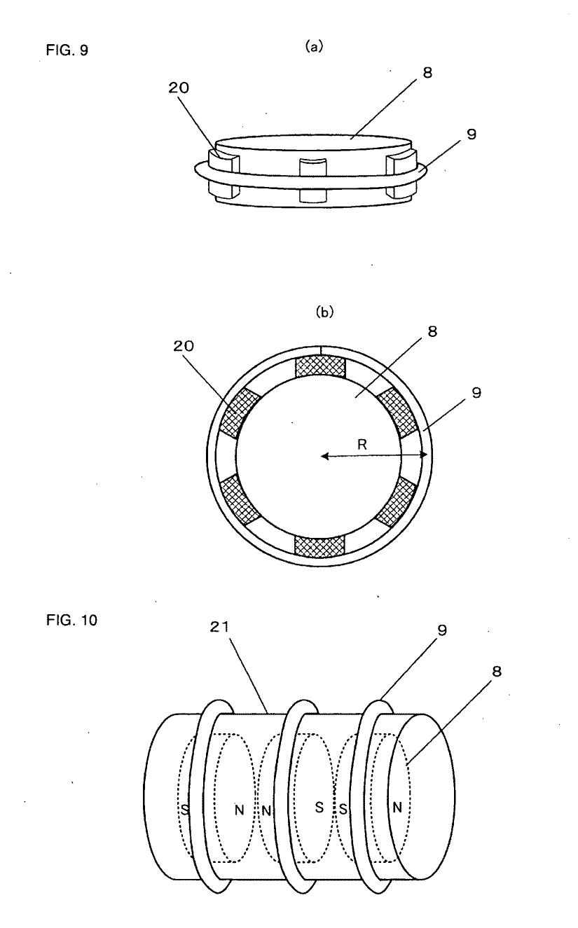

As shown in FIGS. 9A and 9B, when a long-tubule is wound around a magnet 8, it is necessary for the long tubule 9 to be located where the magnetic field generated around the magnet has optimum intensity for plasma to be generated through electronic cyclotron resonance. Therefore, as shown in FIG. 9, spacers 20 are formed of a nonmagnetic material, such as Teflon (registered trademark), around the magnet 8, and the long tubule 9 is wound around the spacers 20, so that the distance R between the long tubule and the center of the magnet can be set to an optimal value.

Here, FIG. 9A is a perspective diagram and FIG. 9B is a plan diagram as viewed from the top.

Furthermore, as shown in FIG. 10, a number of permanent magnets or electromagnets are used as the magnetic field applying means 8, and the magnets 8 are arranged so that the same magnetic poles face each other, and thus, rings of magnetic fields can be generated side by side. Plasma rings are generated around the magnets side by side in the same arrangement as the magnets. Therefore, even when the tubule 9 is long, the long tubule is easy to wind further around the magnets. 21 is a magnet holder formed of a nonmagnetic material.

In addition, as shown in FIG. 12, a number of magnets in ring form 87 are provided as the magnetic field applying means, so that mirror fields are generated in between. In addition, a long tubule 9 is provided in the vicinity of the center of the mirror fields so that plasma can be generated. 88 is a support member or a container member for providing the long tubule 9 in a predetermined location. It is possible to provide a long tubule 9 along the mirror fields, and thus it is possible to provide a straight long tubule, or a folded one, as in FIG. 12.

In addition, in the case where the region where a long tubule is placed is longer than the mirror fields, either the support member 88 or the magnets in ring form 87 are moved (see arrows), so that plasma can be generated throughout the entirety of the long tubule in the configuration.

Here, the same structure can be provided using a solenoid field instead of mirror fields.

(Example of Long Tubule (Object to Be Sterilized))

Long tubules on which the plasma generating device and method according to the present invention are used include objects to be sterilized. Concretely, these are long tubules having an inner diameter of 5 mm or less and a length of 10 cm or more, such as catheters and endoscopes. Though tubules of a variety of materials can be sterilized, they need to be formed of a non-conductive material, and the present invention is particularly effective for long tubules formed of a resin material, such as silicone rubber, polyimide, vinyl chloride, polyurethane or a fluorine resin.

The present invention makes it possible to kill various bacteria clinging to the surface—particularly the inner wall—of a long tubule. The present invention can also decompose and remove lipids and proteins clinging to the inner wall of a catheter or the like.

(Plasma Generating Method)

Next, the plasma generating method is described.

A long tubule is provided in the magnetic field applying means 8, which is placed in the container 1 in advance in FIG. 1.

The pressure reducing means 6 is operated, so that the pressure within the container lowers to 1 Pa or less, preferably 0.01 Pa or less, and remains there.

In the case of a sterilizing process, a gas containing any one of oxygen, argon or air is supplied from the gas supplying means 4 while the pressure within the container 1 is maintained at approximately 0.1 Pa. One of the things that characterizes the present invention is that there is a difference in pressure between the inside and outside of the long tubule. When plasma is selectively generated only inside or outside the long tubule 9, the pressure either inside or outside the long tubule is adjusted to a level appropriate for electronic cyclotron resonance.

In the case where the pressure within the container is adjusted as described above, the pressure inside the long tubule is generally higher than outside, though this depends on various conditions, such as the inner diameter and length of the long tubule.

One way of providing a difference in pressure between the inside and outside of the long tubule is by holding the long tubule in such a state that it is at least partially bent, as in FIGS. 2 to 4. When the long tubule is bent with a small curvature, the fluidity of the gas lowers and the viscosity increases, and therefore it is easy to increase the difference in pressure between the inside and outside of the long tubule.

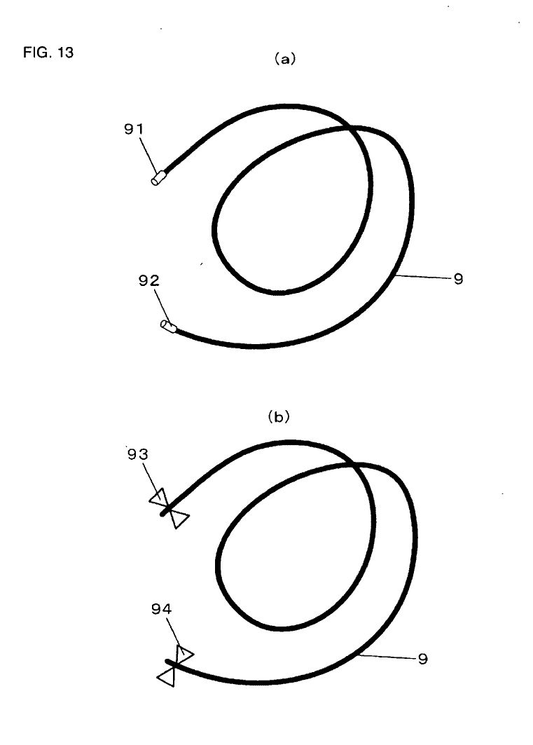

It is also possible to create a difference in pressure by making the opening at the ends of the long tubule narrow. Concretely, as shown in FIG. 13A, the ends of the long tubule can be covered with caps 91 and 92, or the openings may be partly covered with Teflon (registered trademark) tape.

Furthermore, as shown in FIG. 13B, the ends of the long tubule may be pinched with clips 93 and 94 in order to make the openings narrow.

Here, in the case where the two ends of the long tubule are completely sealed with caps or clips, the pressure within the long tubule 9 cannot be adjusted, and therefore some degree of opening is necessary. These methods for narrowing the openings are particularly effective in the case where the inner diameter of the long tubule is as much as 5 mm or more.

The present invention can be applied both when the pressure inside the long tubule is high and when the outside is high. In the case where the mechanical strength of the wall of the long tubule is low, for example, it is difficult to generate plasma when the external pressure is higher than the internal pressure, because the tubule may flatten. In addition, it is necessary to connect a pipe for introducing and discharging a gas to the long tubule in order to make the internal pressure lower than the external pressure.

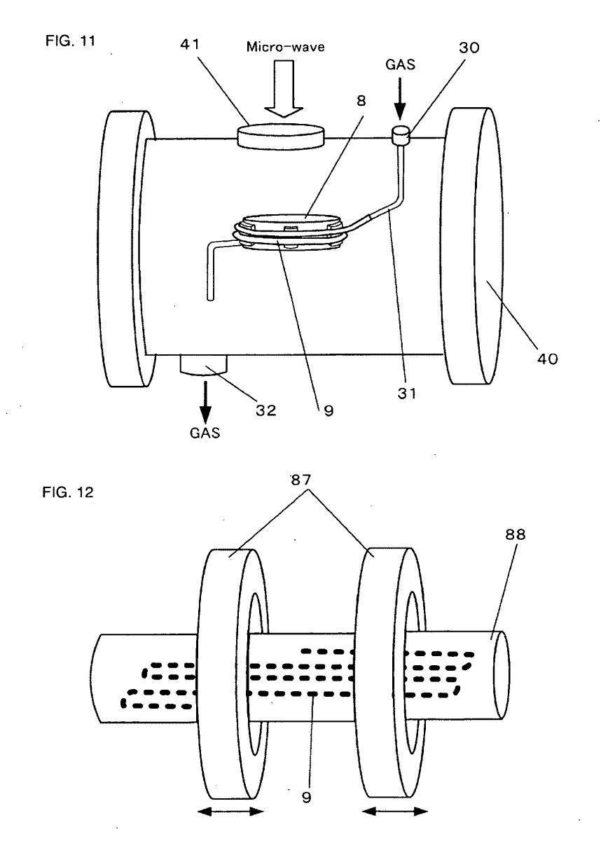

It is absolutely necessary to control the pressure within the long tubule with high precision in order to generate plasma within the long tubule. It is possible to attach a gas pipe 31 at the end of the long tubule so that a gas can be introduced directly into the long tubule, as shown in FIG. 11. In this case, a vacuum pump is connected to a port 32, so that the inside of the vacuum container 40 can be kept at a certain vacuum level while a gas continuously flows into the long tubule 9 through the gas pipe 31 from the gas introducing port 30. In this state, the pressure within the long tubule 9 is easy to adjust, by adjusting the amount of supplied gas. The port 41 is a portion through which microwaves are introduced.

Here, there is a risk that the long tubule may be contaminated by the pipe when a sterilizing process is carried out on the long tubule, and therefore, it is necessary to be extra careful when handling the pipe.

Next, in the case where permanent magnets are used to apply a magnetic field, the magnetic field can be applied to the long tubule simply by placing it in the magnetic field applying means 8. In the case where electromagnets are used, it is preferable to start applying the magnetic field when the inside of the container has reached a certain pressure.

Thus, the long tubule, which is under a certain pressure and to which a magnetic field is applied, is irradiated with microwaves. In this state, plasma is easy to generate through electronic cyclotron resonance.

In the case where a sterilizing process is carried out on a long tubule made of a resin, as described above, it is preferable to set the temperature inside the long tubule to 60° C. or lower.

In order to stably generate plasma with low energy, it is necessary to adjust the input power for the microwave supplying means.

Accordingly, the voltage value, frequency and waveform of the alternating current voltage for driving the magnetron are set taking into consideration the fact that the power required for the generation of plasma is supplied, and it is possible to carry out a sterilizing process without damaging the long tubule with the generated plasma.

The conditions for generating plasma depend on the pressure and type of gas within the long tubule, and various gases, for example oxygen, a mixed gas of argon and oxygen, steam or carbon dioxide can be used. In addition, any value within a range from 0.01 Pa to 1 Pa, in which electronic cyclotron resonance is possible, can be selected for the pressure within the long tubule.

There is a way to adjust the input power by changing the voltage value of the alternating current voltage applied to the microwave generating portion, and there is a way to achieve pulse drive by adjusting the waveform of the alternating current voltage applied to the microwave generating portion. In pulse drive, the ON period t1 and OFF period t2 are adjusted in the pulse waveform P, which is a synthetic waveform of an alternating current waveform W having a predetermined frequency (2.45 GHz) and a pulse waveform P having a longer period than frequency. It is appropriate for the frequency of the pulse waveform (1/(t1+t2)) to be 0.1 pps (pulse per second) to 100 pps. In addition, the greater the value t1/(t1+t2) is, the higher the temperature of the object is, and the greater the value of t2 is, the more difficult regeneration of plasma is. In addition, though in FIG. 7 the maximum value of the pulse waveform P is 1 and the minimum value is 0, a structure where plasma and associated radical atoms do not disappear completely is also possible, when the minimum value to a value within a range of 0 to 0.5, for example.

(Use of Antibacterial Bag)

It is necessary to prevent bacteria and dirt from clinging to long tubules, such as catheters, directly before use, and therefore in some cases they are contained in a resin bag for preventing bacteria from entering.

The resin bag has such properties that bacteria can be prevented from entering but gases can pass through. Concretely, bags made of unwoven sheet where ultra-fine, long fibers made of 100% polyethylene are combined by applying heat and pressure (Tyvek (registered trademark, made by DuPont)) can be used, for example.

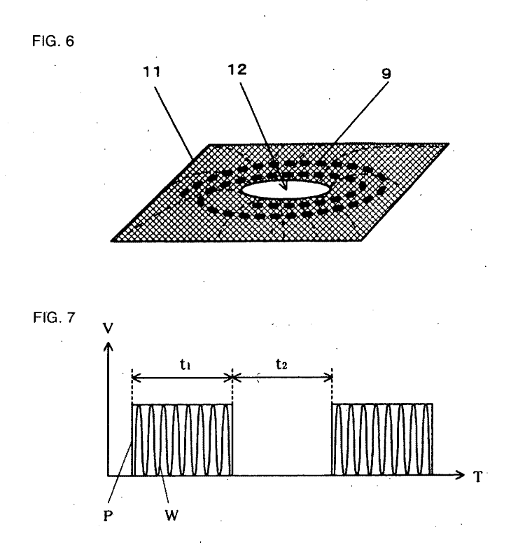

When a long tubule is put in a resin bag (packaging process) after being sterilized, there is a risk that bacteria may cling to the long tubule or get into the resin bag. In order to prevent this, the present invention makes it possible to carry out a sterilizing process with the long tubule 9 in the resin bag 11, as shown in FIG. 6. In addition, the bag 11 has a hole at the center (the bag itself is sealed), so that a magnetic field applying means 8 can be inserted, as shown in FIGS. 1 and 5.

(Combination with Sterilizing Device for External Surface)

Though the main point of the plasma generating device in FIG. 1 is to generate plasma inside the long tubule 1, in some cases it is necessary to sterilize the external surface of the long tubule as well. In such cases, it is necessary to generate plasma and oxygen radicals outside the long tubule 1 within the container 2. Various methods can be used to generate these, and one example is the method using high frequency waves (RF) and an antenna in Patent document 3, for example.
Patent Document 3: Japanese Unexamined Patent Publication 2006-20950

### EXAMPLES

An experiment was conducted using a long tubule (made of silicone rubber) having an inner diameter of 2 mm and a length of 50 cm in the plasma generating device in FIG. 1.

The long tubule was bent to a circular form and placed around the cylindrical permanent magnet (made of neodymium, having a diameter of 3 cm, a height of 1.5 cm, and a surface magnetic field of 4000 G), which was fixed to a Teflon (registered trademark) plate.

The long tubule and the permanent magnet were both placed in the container 1. The air within the container was replaced with an oxygen gas and the pressure within the container adjusted to 0.1 Pa, and then microwaves of 4.5 GHz were emitted into the container 1.

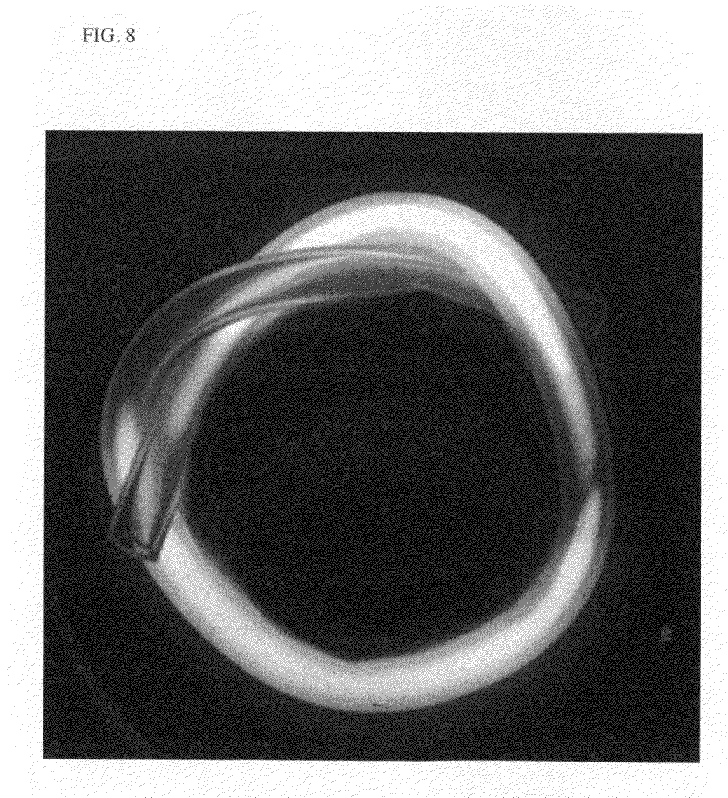

FIG. 8 is a photograph showing how glow discharge was generated only inside the long tubule. It could be confirmed that plasma was generated throughout most of the long tubule. It is clear from this that plasma is easy to generate within a long tubule through electrodeless discharge according to the present invention.

Next, an experimental device was manufactured, as shown in FIG. 11, and a sterilizing process was carried out on a long tubule (having an inner diameter of 5 mm and a length of 100 cm).

In order to confirm the state of sterilization, a biological indicator (for Geobacillus stearothermophilus, made by Raven Corporation) was placed inside the long tubule 9. For the experiment, the pressure within the vacuum container 40 was kept at 6.0×10⁻² Pa to 9.0×10⁻² Pa, and the microwave output was 200 W to 800 W. Air was used as the gas.

The microwaves were supplied in pulses, as shown in FIG. 7, and whether the long tubule was completely sterilized was checked over the total time of irradiation with plasma. It could be confirmed that the long tubule was completely sterilized after being irradiated with plasma for 5 seconds or longer.

### INDUSTRIAL APPLICABILITY

As described above, according to the present invention it is possible to provide a plasma generating device and method for generating plasma through electrodeless discharge within a long tubule, so that a plasma process is carried out inside the long tubule.

1. A plasma generating device, comprising:
a container for a long tubule that is open at both ends, wherein pressure inside of the long tubule is adjustable;
a magnetic field applying means for applying a magnetic field in at least part of the long tubule; and
a microwave supplying means for emitting microwaves into the container, wherein
microwaves are emitted into the container in such a state that at least one end of the long tubule is inside the container, the pressure inside the long tubule is adjusted to a predetermined pressure that is higher than a pressure within the container, and a magnetic field is applied in at least part of the long tubule so that plasma is generated within the long tubule.

2. The plasma generating device according to claim 1, wherein the magnetic field applying means is formed of at least a permanent magnet or an electromagnet.

3. The plasma generating device according to claim 2, wherein the magnetic field applying means is formed of a number of permanent magnets or electromagnets, and the magnets or electromagnets are arranged in such a manner that same magnetic poles face each other.

4. The plasma generating device according to claim 1, further comprising pressure adjusting means for adjusting the pressure inside the container, configured to reduce the pressure within the container to 1 Pa or less.

5. The plasma generating device according to claim 1, wherein the long tubule is an object to be sterilized using the plasma.

6. The plasma generating device according to claim 5, wherein a gas containing at least one of oxygen, argon and air is introduced into the container.

7. The plasma generating device according to claim 1, wherein the long tubule is an object to be coated with a film or etched on an inner wall of the long tubule using the plasma.

8. The plasma generating device according to claim 1, wherein a gas is introduced directly into the long tubule.

9. A plasma generating method, comprising:
putting a long tubule that is open at both ends in a container; and
emitting microwaves into the container in such a state that at least one end of the long tubule is inside the container, an inside of the long tubule is adjusted to a predetermined pressure that is higher than a pressure within the container, a magnetic field is applied in at least part of the long tubule, and plasma is generated inside the long tubule.

10. The plasma generating method according to claim 9, wherein the pressure inside or outside the long tubule is adjusted so that there is a predetermined difference in pressure between the inside of and an outside of the long tubule.

11. The plasma generating method according to claim 10, wherein making the predetermined difference in pressure comprises: holding the long tubule in such a state that at least part of the long tubule is bent; or making an opening at an end of the long tubule narrow.

12. The plasma generating method according to claim 9, wherein at least part of a magnetic field applying means for applying a magnetic field is provided inside the container and the long tubule is provided so as to be wound around part of the magnetic field applying means.

13. The plasma generating method according to claim 9, wherein the magnetic field is a solenoid field or a mirror field, and the long tubule is placed in a vicinity of a center of the magnetic field.

14. The plasma generating method according to claim 9, wherein the pressure within the long tubule is 0.01 Pa to 1 Pa when plasma is generated.

15. The plasma generating method according to claim 9, wherein the long tubule is an object to be sterilized.

16. The plasma generating method according to claim 15, wherein a gas containing at least one of oxygen, argon and air is introduced into the container.

17. The plasma generating method according to claim 15 wherein input power for the microwaves is adjusted so that a temperature inside the long tubule remains at 60° C. or lower.

18. The plasma generating method according to claim 15, wherein the long tubule is contained in a resin bag for preventing bacteria and viruses from entering.

19. The plasma generating method according to claim 9, wherein the long tubule is an object to be coated with a film or etched using plasma, on an inner wall surface of the long tubule.

20. The plasma generating method according to claim 9, wherein a gas is introduced directly into the long tubule.
